(19) United States
(12) Patent Application Publication
(10) Pub. No.: US 2017/0203432 A1
(43) Pub. Date: Jul. 20, 2017

(54) ACTUATION APPARATUS FOR WEARABLE DEVICES

(71) Applicant: Konstantinos Andrianesis, Corinth (GR)

(72) Inventor: Konstantinos Andrianesis, Corinth (GR)

(21) Appl. No.: 15/407,003

(22) Filed: Jan. 16, 2017

Related U.S. Application Data
(60) Provisional application No. 62/279,733, filed on Jan. 16, 2016.

(51) Int. Cl. B25J 9/00; A61F 2/68; B25J 13/08; A61H 1/02; B25J 9/10; B25J 9/16; A61F 2/54; A61F 5/01

(52) U.S. Cl. CPC B25J 9/0006; A61F 2/54; A61F 2/68; A61F 5/013; A61H 1/0274; B25J 9/104; B25J 9/1075; B25J 9/1694; B25J 13/081; A61F 2002/5093

(57) ABSTRACT

An apparatus for use in actuating a wearable terminal device includes a housing coupled to a harness system, a control unit, a movable actuating element having its one end in the housing and the other end connected through coupling means to the terminal device, and a conduit which contains the actuating element. The actuating element is a body-powered cable, an artificial tendon coupled to an actuator device installed in the housing, or an actuator wire made of smart material.

### CROSS-REFERENCE TO RELATED APPLICATIONS

The present application claims the benefit of U.S. Provisional Patent Application No. 62/279,733, filed on Jan. 16, 2016, which is incorporated herein by reference in its entirety.

### FEDERALLY SPONSORED RESEARCH

Not Applicable

### SEQUENCE LISTING OR PROGRAM

Not Applicable

### BACKGROUND

The present invention is in the technical field of wearable assistive devices.

Wearable assistive devices such as prosthetic, orthotic, and empowering devices are commonly used to increase, maintain, or improve functional capabilities of individuals with upper-limb disabilities. Many sophisticated devices have already been publicly demonstrated. For example, in the prosthetics field, the state-of-the-art devices include myoelectrically controlled motorized prosthetic hands with individually movable fingers, capable of performing various grip patterns. These terminal devices are intrinsically actuated by using direct-current (DC) electric motors housed within their volume (palm and fingers). A custom fabricated prosthetic socket is closely fitted to the amputated limb of each user. Myoelectrodes embedded in the socket sense user intention and control the prosthesis. Energy supply is provided from a rechargeable battery close by the terminal device and typically integrated in the socket.

However, these active prosthetic devices have a number of shortcomings, including being slow, heavy, large, and noisy, thus leading to low user satisfaction and acceptance rates. All these issues are strongly related with the limitations imposed by the use of conventional DC motors and their associated driving systems, as well as the size constraints imposed by the hand envelope dimensions wherein the motors are located; it is well known that DC motors undergo drastic reductions in power as they are scaled down and as a consequence, bulky, heavy and noisy driving systems (typically complex gearing systems) are requisite. Moreover, since all the motors are housed within the hand envelope, prosthesis feels unpleasantly heavy to the user owing to its bad center of gravity and weight distribution. The increased weight also leads to the need for a more firmly attached socket that limits elbow motion and also results in pain and discomfort symptoms to the user. Therefore, most of the amputees either totally abandon this type of externally-powered prosthetic devices, or prefer to minimize its usage time and use other prostheses options, such as body-powered prostheses, for most of their daily tasks.

Body-powered prosthetic devices use a Bowden-cable actuation system, wherein a control cable, coupled to a harness that is worn by the user, activates the terminal device (hook or hand) by means of gross body movements (e.g., scapular abduction, shoulder elevation, elbow flexion). These devices are usually either voluntary closing devices, wherein the force generated through the harness acts to close the terminal device and perform a grip, or voluntary opening devices that are opened at will by the user while biasing springs or elastic bands are used to close the terminal device and perform a grip. The benefits of body-powered devices include their low weight, quick intuitive response, robust construction, silent operation, reliability, battery independency, and a degree of proprioceptive force and position feedback to the user through the mechanical interaction between the harness and control cable; particularly in voluntary closing devices, the latter feature provides the user with a direct and intuitive relation between actuation force and pinch force. On the other hand, the force that the user must usually exert to operate the terminal device is high, causing fatigue and repetitive strain injuries to the amputees. Additionally, wearers typically complain that these systems are uncomfortable due to the restrictive harness, the unnatural body movements required to be operated, and the need for a high range of motion to function properly. Moreover, since available body movements for prosthesis control are limited, usually only one or two degrees of freedom can be controlled. Therefore, as opposed to multi-articulated motorized hands, the body-powered devices are mostly used as simple grippers (open/close function) without any dexterous functions.

As in prosthetics, other commonly used assistive upper-limb devices, such as orthotic and empowering devices, remain less than optimal for satisfying user needs, mostly due to the aforesaid issues of the utilized actuation systems. Many people are in need of orthotic devices to restore their hand's lost or weak functions (e.g., following a disease or a neurological condition) to their natural levels, or of empowering devices to extend the strength of their hand beyond its natural ability (e.g., elderly people, astronauts, manual workers in certain situations). Several such wearable devices have already been known in the art, including robotic exoskeletons and gloves. But features like portability, weight, functionality, comfort, and user interface of such devices must be significantly improved to increase their applicability and practicality.

Thus, there remains a considerable need for actuation systems for wearable assistive upper-limb devices with improved features, including being compact, lightweight, with increased functionality, comfortable, and user-friendly.

### SUMMARY

The present invention is directed to actuation systems for wearable upper-limb devices such as prosthetic, orthotic, empowering, or other assistive upper-limb devices. It is an aim of this disclosure to provide an actuation apparatus that addresses issues associated with the known art. This apparatus exploits an extrinsic actuation concept wherein driving devices are installed remotely and not within the volume of such a wearable upper-limb device (in the following referred to as “terminal device”).

According to a first aspect of the present invention, this apparatus includes a housing coupled to a harness system that can be worn by a user, a control unit, one or more movable actuating elements for driving the terminal device having their one end fixed in the housing and their other end connected through coupling means to the terminal device, and a conduit that contains these actuating elements. Each actuating element may be a body-powered cable, an artificial tendon coupled to an actuator device installed in the housing, or an actuator wire made of smart material, preferably of shape-memory material.

These actuating elements are preferably enclosed by low-friction flexible tubes in order to minimize friction losses. Additionally, in preferred embodiments, the housing comprises a length-adjusting mechanism for altering the setup position of the actuating elements and conduit, and thus enabling the actuation apparatus to fit different-sized users. In yet another aspect of preferred embodiments, position and tension sensors for the actuating elements are used to provide feedback. Still more preferred embodiments additionally include a user interface that comprises a plurality of user input and feedback options.

Various objects, features, aspects, and advantages of the present invention will become more apparent from the following detailed description of preferred embodiments of the invention, along with the accompanying drawing figures in which like numerals represent like or similar components.

### BRIEF DESCRIPTION OF THE DRAWING FIGURES

FIGS. 1-3 are perspective views of an actuation apparatus of a prosthetic hand worn by an amputee.

FIG. 4 is a schematic top-view diagram of components disposed within the housing of the actuation apparatus shown in FIGS. 1-3.

FIG. 5 is a block diagram of the control architecture of the actuation apparatus shown in FIGS. 1-3.

FIG. 6 is a schematic side-view diagram of the coupling means used for connecting the actuation apparatus shown in FIGS. 1-3 to the prosthetic hand.

FIG. 7 is a perspective view of the user interface of the actuation apparatus shown in FIGS. 1-3.

### DETAILED DESCRIPTION

The following description is only illustrative of the present invention and is made merely for the purpose of providing a full and enabling disclosure of the invention. Possible preferred embodiments will be described with reference to the drawing figures, wherein like numerals refer to like or similar components throughout the several figures, and those skilled in the art will understand that alternative configurations and combinations of components may be substituted without departing from the substance or scope of the present invention. Also, in some figures, details that are not necessary for an understanding of the invention or that render other details difficult to perceive may be omitted.

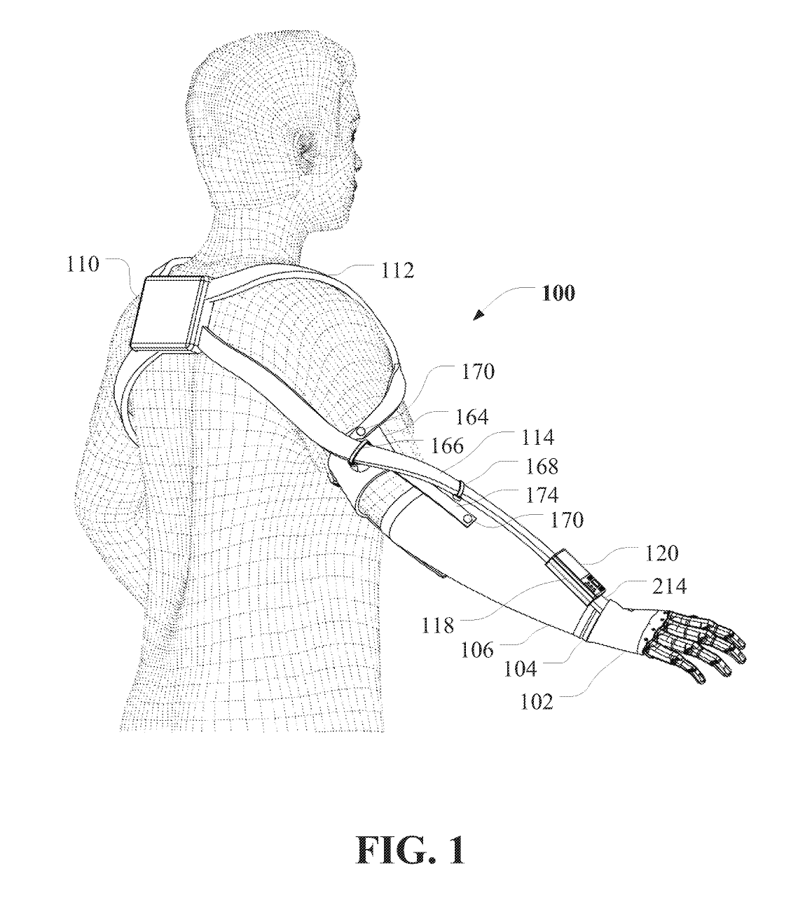

Referring to FIGS. 1-3, an illustrative embodiment of the present invention is shown in different perspective views. Specifically, an actuation apparatus 100 is used for actuating a terminal device 102 and particularly a prosthetic hand worn by a unilateral transradial amputee. The terminal device 102 is attached to the stump of the amputee by means of a standard wrist unit 104 and a prosthetic socket 106. The actuation apparatus 100 includes a housing 110 (shown in FIG. 1) coupled to a harness system 112 that is worn by the amputee, and a conduit 114 that contains movable actuating elements for driving the terminal device 102, having their one end fixed within the housing 110 and their other movable end connected through coupling means 118 to the terminal device 102. The actuation apparatus 100 may further comprise a user interface 120 that will be discussed in detail below.

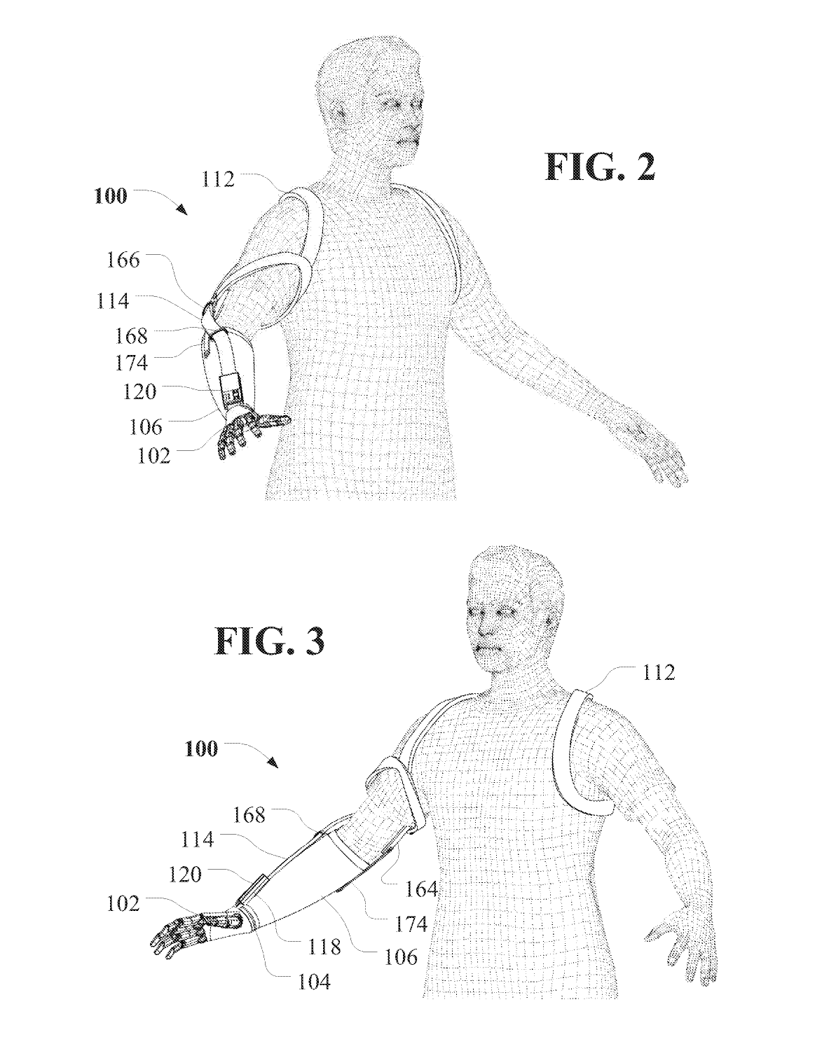

Referring to FIG. 4, a schematic top-view diagram is illustrated including components that are preferably disposed within the housing 110. As previously mentioned, the one end of the movable actuating elements is fixed in the housing 110. These movable actuating elements may be body-powered or externally powered. Specifically, a movable actuating element may be a body-powered cable 122, an artificial tendon 124 coupled to an actuator device 128 installed in the housing 110, or an actuator wire 126 made of smart material. The two different terms “cable” and “tendon” are used herein only for reference purposes to promptly discriminate the different types of actuating elements of the actuation apparatus 100, and in no way intended to imply material differentiation. The body-powered cable 122 and the artificial tendon 124 may be made of any suitable material, preferably of a strong, durable, low-friction and flexible material. For example, nylon-coated multi-strand stainless steel wires may be used towards this purpose.

FIG. 4 is only illustrative of the different types of the actuating elements that may be used. One or more actuating elements of any of the three aforesaid types or any combination thereof may be used. Thus, it is to be understood that there are numerous possible embodiments of the actuation apparatus 100 with regard to the type and number of its actuating elements. For example, an embodiment of the present invention may include only the body-powered cable 122. Other embodiments may comprise a plurality of actuator wires 126, artificial tendons 124 along with corresponding actuator devices 128, or both. In further embodiments, the actuation apparatus 100 may include a hybrid actuation system, wherein both body-powered and externally powered actuating elements are used. For example, in such embodiments, the terminal device 102 may be operated by means of the body-powered cable 122 and body movements of the user, and, in addition, a plurality of actuator wires 126 and/or artificial tendons 124 along with corresponding actuator devices 128 may be used to automatically provide force-assistance to the user (e.g., to perform and maintain a grip).

In general, the actuating elements are used for driving the terminal device 102, but the specific function of each actuating element may differ. For example, an actuating element may be used for actuating one or more degrees of freedom of the terminal device 102, locking the motion of one or more degrees of freedom of the terminal device 102, or selecting mechanically an operating mode of the terminal device 102 (e.g., a grip or a set of grips).

Still referring to FIG. 4, terminals 132-136 are used for crimping the one end of the corresponding actuating elements 122-126. The terminals 132, 136 are fixed to a support plate 130, and the terminal 134 is fixed to the actuator device 128 which in turn is fixed to the support plate 130, by means of screws or any other suitable fastening means.

The actuating elements 122-126 are disposed and freely movable within the conduit 114. The one end of the conduit 114 is fastened to the support plate 130 by means of a conduit mount (not shown) of the type known in the art. The conduit 114 is preferably made of a flexible, durable material including but not limited to plastic or woven materials. The structure of the conduit 114 may also vary from a simple tube or cable sheath to a more complex multi-body articulated structure similar to the one used in endoscopes and well known to people of ordinary skill in this art. Along the length of the conduit 114, guide parts (not shown) may be used to keep the actuating elements 122-126 in predetermined paths within the conduit 114, so that they do not influence each other's motion. Furthermore, in order to minimize friction losses and improve the efficiency of the actuation apparatus 100, the actuating elements 122-126 preferably follow a path without large angle bends.

Additionally, low-friction flexible tubes 138, for example, narrow-gauge tubes made of PTFE, may enclose each of the actuating elements 122-126 to reduce friction losses. Pins 140 or like may also be used as route guides of one or more of the actuating elements 122-126.

In some preferred embodiments, the actuator device 128 is a standard, commercially available, linear DC motor whose actuation direction is shown by the arrow 162. The actuator device 128 preferably includes a position feedback sensor (e.g., a potentiometer or an encoder). To reduce vibration and noise, the actuator device 128 may be disposed on flexible mounts (not shown). To further reduce noise, the actuator device 128 may be encased in a soundproofing material. To further reduce noise, the actuator device 128 may have a motor without a gearbox.

Alternatively, the actuator device 128 may be of any known rotational or linear actuator type, including but not limited to DC motors, AC motors, stepper motors, servo motors, ultrasonic motors, solenoids, pneumatic actuators, hydraulic actuators, and piezoelectric actuators. In further embodiments, the actuator device 128 may be a device comprising any smart material which is capable of changing its shape upon application of a relevantly appropriate form of energy, including but not limited to electrostrictive polymers, electroactive polymers, conductive polymers, electrostatic devices, piezoelectric polymers, mechano-chemical polymers and gels, shape-memory polymers, and shape-memory alloys.

The actuation apparatus 100 may be electrically supplied by any type of power source, including but not limited to a battery, fuel cell, solar cell, regulated DC power supply, and mains AC power source. In preferred embodiments, a power source 160 is disposed in the actuation apparatus 100. The power source 160 is preferably a portable, rechargeable battery or battery pack of high energy capacity. Furthermore, the power source 160 is preferably installed in an easy access and removable compartment of the housing 110.

In preferred embodiments, the housing 110 further includes a control unit 150. Alternatively, the control unit 150 and/or the power source 160 may be disposed in other locations of the actuation apparatus 100 or externally in other devices, preferably portable, that interface with the actuation apparatus 100, either worn or carried near the user (e.g., clipped on clothing, belt, or carried in a bag or pack).

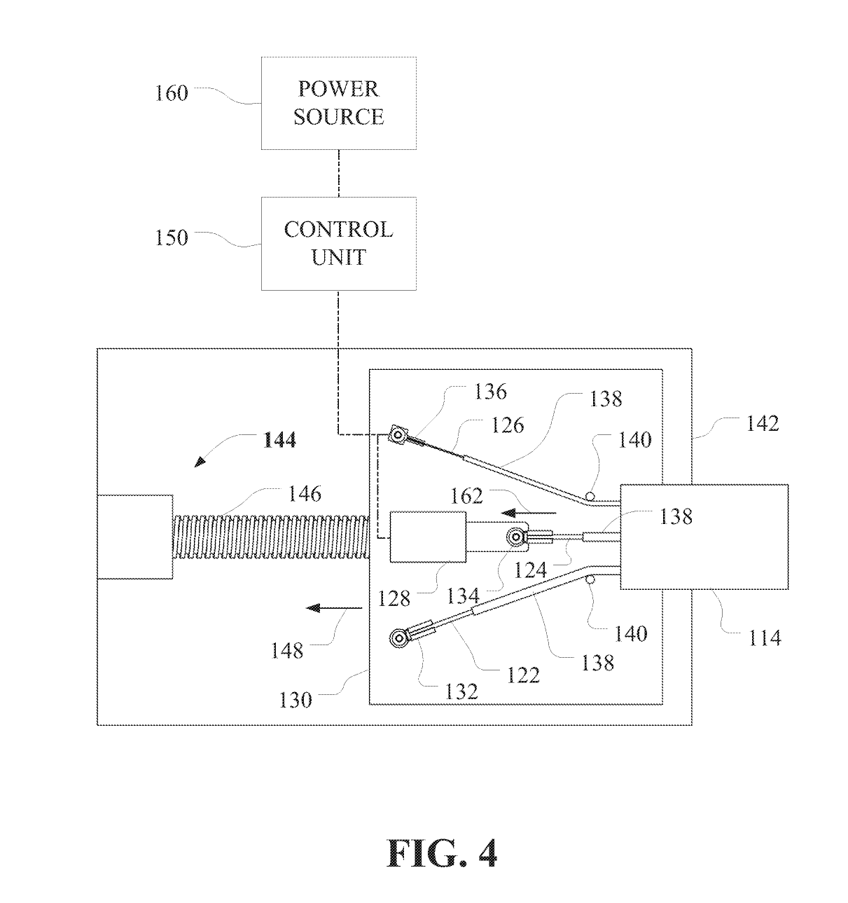

Referring to FIG. 5, a block diagram of the control architecture of the actuation apparatus 100 is illustrated. Specifically, the control unit 150 preferably includes suitable electronics known in the art to drive the actuators 152, including the actuator device 128 and the actuator wire 126, by sending the required control signals, receive feedback data from the actuators 152, collect or receive data from a plurality of sensors 154 that will be discussed in detail below, process the received data from the sensors 154, interface with the terminal device 102 and the user interface 120, including user input 156 and user feedback 158 devices. Additionally, the control unit 150 may include suitable electronics to communicate to a personal computer, a tablet computer, a smartphone, or other terminal devices, via standard communication protocols known in the art. The communication channels of the actuation apparatus 100 may be implemented either by wires (e.g., via TWI, SPI, UART protocols) or wirelessly (e.g., via RF, Bluetooth, WiFi, ZigBee protocols).

The sensors 154 may include electrical power sensors to measure voltage, current, and/or power levels of the actuators 152 and/or the power source 160. Additionally, the sensors 154 may include temperature sensors for monitoring the ambient temperature at specific parts of the actuation apparatus 100 and/or the temperature on the actuators 152, and thus mitigating the associated risks of extreme temperature conditions that may lead to hardware failure and/or user harm. Apart from temperature sensors, temperature detection may be provided by using a thermochromic polymer material, which changes color due to temperature change. Other ambient conditions sensors (e.g., humidity sensors) may also be included in the actuation apparatus 100. As it will be discussed in the following, the actuation apparatus 100 may also include other types of sensors configured to detect information about the operation and status of the actuation apparatus 100, the terminal device 102, the user, and/or the user's environment.

Referring again to FIG. 4, the housing 110 may further comprise a length-adjusting mechanism 144, mounted on a base plate 142, for altering the setup position of the support plate 130, and thus the position of the actuating elements 122-126 and of the conduit 114. With such a mechanism, the actuation apparatus 100 may be easily adjusted to fit various arm sizes. The mechanism 144 may be a simple lead-screw mechanism, well known to people of ordinary skill in this art. When turning a lead screw 146, the support plate 130, which is coupled to a traveling nut (not shown), moves linearly in the direction of the arrow 148, back or forth depending on the direction of rotation of the screw 146. Other linear positioning mechanisms with self-locking attributes may also be used towards this scope, including not only manually operated mechanisms, but also actuator-operated ones. Although not shown, similar length-adjusting mechanisms of smaller dimensions may also be used for adjusting the setup position of the terminals 132-136 of each of the movable actuating elements 122-126, in order to compensate for any creep or loosening of the actuating elements 122-126.

The plates 130, 142 are preferably constructed from a strong, durable, and lightweight material (e.g., nylon, carbon fiber, aluminum). The support plate 130 may also be a printed circuit board with the necessary wiring for interfacing the actuators 152 with the control unit 150.

In some preferred embodiments, the actuator wire 126 is made of a shape-memory alloy, and specifically a specially processed Nickel-Titanium (Ni—Ti) alloy, commercially available by several manufacturers (e.g., one-way Flexinol® actuator wire by Dynalloy Corporation). Due to a dynamic change of its internal structure at certain temperatures, the actuator wire 126 has the ability to contract (typically about 4% of its length) when heated and can then be easily stretched out again as it cools back to room temperature. During its activation, if it encounters any external resistance, it can yield ample actuation force. This type of actuator presents an incredibly high power-to-weight ratio and totally silent operation.

The actuator wire 126 can be easily driven via electrical resistance heating. This can be done by using any type of electrical current (DC or AC). In more preferred embodiments, pulse width modulation (PWM) method is used for driving the actuator wire 126. PWM activation is beneficial as it enables a uniform heating of the actuator wire 126 with little generated waste heat, thereby resulting in improved energy efficiency. PWM activation is also preferable because of the ease with which it can be implemented using microcontrollers in the control unit 150. Alternatively, other heating methods, well known to people skilled in the art, may be used for driving the actuator wire 126 (e.g., using a fluid as a heating/cooling medium).

An additional characteristic of such a shape-memory alloy wire 126 is that its electrical resistance decreases as it is heated and contracts. This relationship between electrical resistance and strain can be used for position feedback of the actuator wire 126. By monitoring its electrical resistance, a good estimation of the contraction level can be obtained. The electrical resistance can be easily calculated by measuring the voltage drop and electrical current flowing in the actuator wire 126. All these measurements and calculations may be carried out by the control unit 150. Additionally, various control schemes, including linear and nonlinear control schemes, may be implemented in the control unit 150 for controlling the actuator wire 126.

As this actuator type is thermomechanical in nature, its cooling rate through natural convection is much slower than its heating one. The cooling rate of the actuator wire 126 is largely determined by heat transfer through its surface. Thus, the smaller the diameter of the actuator wire 126, the higher its surface-to-mass ratio and hence the faster it can cool. Apart from using a smaller diameter actuator wire 126, other methods may be used to improve the speed of cooling of the actuator wire 126, including but not limited to forced air, heat sinks, and liquid coolants.

Although a thinner actuator wire 126 is faster, it is also weaker. Thus, if higher force output is needed, in order to not sacrifice actuation speed, multiple actuator wires 126 may be used in a work-summation layout known in the art. For example, a plurality of actuator wires 126 may be mechanically bundled, in spaced parallel relation to one another and thus out of contact with one another, so that a cooling medium (e.g., air) can evenly dissipate heat from all wire surfaces.

Owing to the low strain capabilities of this type of actuator, the actuator wire 126 needs to be long enough to generate a sufficient contraction stroke. The actuation apparatus 100 offers the space needed to accommodate a relatively long actuator wire 126. Furthermore, a much longer actuator wire 126 may be housed within the actuation apparatus 100 by using stroke-multiplying mechanisms well known in the art (e.g., lever or pulley mechanisms). These mechanisms may be preferably implemented within the housing 110.

As previously mentioned, the actuator wire 126 is preferably made of a Ni—Ti shape-memory alloy. Alternatively, the actuator wire 126 may be made of any other shape-memory alloy, or any other shape-memory material (e.g., a shape-memory polymer), or generally any other smart material with similar actuation properties. Alternatively, instead of wire form, this smart-material based actuator may be in a different physical form including, for example, ribbon, sheet, or spring.

Referring again to FIGS. 1-3, the harness system 112 is a shoulder harness that includes straps or like and fits across the shoulders of the user. The coupling between the housing 110 and the harness system 112 may be achieved by any suitable means (not shown) known in the art. In preferred embodiments, this coupling can be reversible to accommodate users with either right or left upper-limb amputation. The harness system 112 may include tightening means (e.g., buckles, friction-type strap attachments) for adjusting straps as needed to fit different-sized users. Alternatively, the harness system 112 may be of any other type that can be used for mounting the housing 110 to the user's back or to any other suitable user's body part, including, for example, a belt, a cuff, or a harness jacket. Harness systems are well known to people of ordinary skill in this art and therefore no further discussion is required within the context of the present disclosure.

The dimensions of the housing 110 may greatly vary depending on the size and/or the distribution of the contained components. In preferred embodiments, the housing 110 is a low-profile design that can fit under clothing. In further embodiments, the actuation apparatus 100 may include a plurality of housings for containing the necessary components, and these housings may be electrically interfaced.

In order to have the body-powered cable 122 work properly, the conduit 114 along with the other containing actuating elements 124-126 is preferably routed as shown in FIGS. 1-3. A triceps cuff 164 along with a retainer 166 is used to anchor the conduit 114 on the back of the user's arm, between the elbow and the shoulder of the user. A second retainer 168 attached on the socket 106 is also used for route guiding of the conduit 114. Fasteners 170 and hinges 174 are used for coupling the triceps cuff 164 to the socket 106 and to the harness system 112. The hinges 174 may be flexible (e.g., straps or like) or rigid (providing more structure and support or for a very short residual limb).

The user may apply tension to the body-powered cable 122 and provide the necessary excursion to actuate the terminal device 102 by harnessing body movements including, for example, glenohumeral flexion, scapular abduction or adduction, shoulder depression and elevation, and chest expansion.

Alternatively to the body-powered cable 122, a hydraulic cylinder system known in the art may be included in the actuation apparatus 100 for reducing friction losses. Such a system includes a master cylinder which extends by harnessing body movements, and actuates through hydraulic hose one or more slave cylinders that can be used for actuating the terminal device 102.

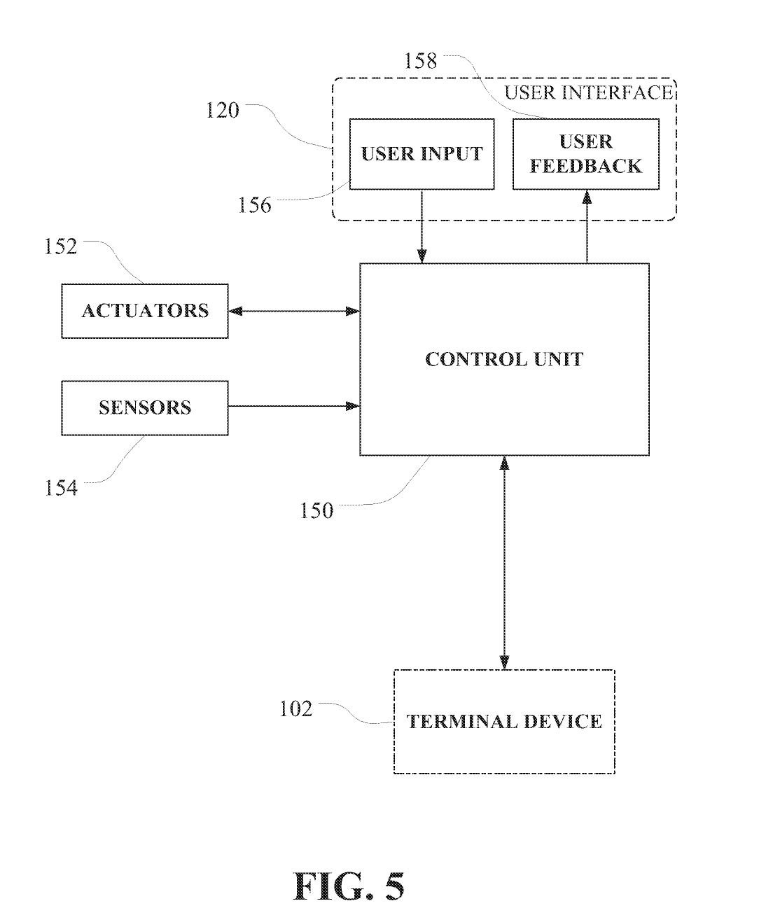

Referring to FIG. 6, the internal assembly of the coupling means 118 is shown in an illustrative side-view diagram. In order to drive the terminal device 102, each movable actuating element 116 originated from the housing 110—wherein actuating element 116 may be any of the three aforesaid types of actuating elements: the body-powered cable 122, the artificial tendon 124, or the actuator wire 126—couples via the coupling means 118 to a respective movable actuating element 216 terminated in the terminal device 102. The actuating element 216 is an artificial tendon, with structural properties like or similar to the ones of the artificial tendon 124, and whose one end is attached to a specific anchor point of the terminal device 102 via suitable coupling means (not shown). In a preferred embodiment, the coupling means 118 comprise two interlocked assemblies, and particularly a first connector assembly 180 and a second connector assembly 280, which includes the components with reference numbers 214-298. The connector shells 182, 282 house the components of the connector assemblies 180, 280, respectively.

The assembly of FIG. 6 shows only the means needed for coupling a first actuating element 116 originated from the housing 110 to a second actuating element 216 that terminates in the terminal device 102. It is readily apparent to anyone skilled in the field that similar means can be used for coupling all the actuating elements originated from the housing 110 to respective actuating elements terminated in the terminal device 102, and can all be housed within the same connector shells 182, 282.

Alternatively or additionally, in order to drive the terminal device 102, suitable coupling means (not shown) may be used to couple the movable end of the actuating elements originated from the housing 110 directly to specific anchor points of the terminal device 102.

The actuating elements 116, 216 are enclosed by the flexible low-friction tubes 138, 238 and further enclosed by the conduits 114, 214, respectively. The connector shells 182, 282 have openings that are preferably slightly larger than the outside dimensions of the conduits 114, 214 to allow a snug fit without impinging the conduits 114, 214. The conduits 114, 214 are fastened to the connector shells 182, 282 by suitable conduit mounts (not shown).

Describing FIG. 6 in further detail, the first actuating element 116 is coupled to the second actuating element 216 by means of the joining parts 186, 286, 288 that move only longitudinally via guides (not shown) that constrain their motion (e.g., guiding grooves). Specifically, the end of the first actuating element 116 is crimped to a terminal 184 that is coupled to the first joining part 186. The end of the second actuating element 216 is also crimped to a terminal 284 that is coupled to the second joining part 286. The way the terminals 184, 284 are coupled to the joining parts 186, 286 is not shown as it will be readily apparent to anyone skilled in the field. For example, screws or like fasteners may be used for this scope.

The second joining part 286 is coupled to the third joining part 288. The third joining part 288 is in contact with the first joining part 186 and transfers the motion and force of the first actuating element 116 to the second actuating element 216. A tension sensor 290, which is coupled to its one end to the second joining part 286 and to its other end to the third joining part 288, measures the tensile force that is transmitted via the second actuating element 216 to the terminal device 102. In some preferred embodiments, tensile force is measured using a concept based on Hooke's law, whereby the extension of a spring, placed in series with the actuating element 216, is measured using a position sensor (e.g., a potentiometer or a hall-effect sensor). Alternatively, the tension sensor 290 may be a force sensor (e.g., strain gauge or force sensing resistor) in a suitable arrangement known in the art.

In other embodiments, the tension sensor 290 may not be included in the coupling means 118, and the joining parts 286, 288 may be implemented as a single part. In further embodiments, the tension sensor 290 may be installed within the housing 110 to measure the exerted tensile force of the actuating element 116 at its fixed end; in such case, although the measured tensile force includes the frictional force due to the sliding of the actuating element 116 within its associated tube 138, it can provide an indicative load measurement of the actuating element 116 that can be used as feedback.

Within the coupling means 118, a position sensor 192 is also preferably included to measure the stroke of the first joining part 186 and thus the motion output of the actuating element 116. The position sensor 192 is preferably a membrane potentiometer with an ultra-thin, lightweight profile. Alternatively, the position sensor 192 may be of any other suitable sensor type (e.g., a magnetic hall-effect sensor) installed in an appropriate arrangement.

Biasing elements (e.g., springs or rubber bands) may be used for moving the joining parts 186, 286, 288 in the opposite direction from the one caused by powering the actuating element 116, and for keeping the actuating elements 116, 216 stretched. For example, in a preferred embodiment, an extension spring 194 has its one end fixed in the first connector assembly 180 and its other end coupled to the first joining part 186, and additionally a compression spring 294 installed in the second connector assembly 280 is biased to the active motion of the joining part 288.

Mechanical stops 196, 296 may also be included in connector assemblies 180, 280 for limiting the travel of the joining parts 186, 286, respectively.

Printed circuit boards 198, 298 installed in the connector assemblies 180, 280, respectively, along with the necessary electronics (not shown) may be used for interfacing with the feedback sensors found in the connector assemblies 180, 280 such as the position sensor 192 and tension sensor 290, as well as the electronics of the user interface 120, control unit 150, and/or terminal device 102. Suitable electrical connectors (e.g., spring contact connectors) known in the art, may be mounted on these circuit boards 198, 298 for exchanging power and communication signals between the connector assemblies 180, 280.

Furthermore, the connector assemblies 180, 280 preferably include a snap-action or like latching mechanism (not shown) for securing the connection between them and facilitating their separation when necessary. Such mechanisms are well known to people of ordinary skill in the pertinent art, and therefore no further relevant discussion is provided in the present disclosure. A sensor (not shown) for detecting whether the connector assemblies 180, 280 are interlocked or not may also be integrated in the coupling means 118 (e.g., a tactile switch or a hall-effect sensor).

The coupling means 118 may further comprise a position locking mechanism (not shown), well known to people skilled in the art, for holding the position of the actuating element 116 without requiring constant power consumption.

In further embodiments, within the coupling means 118, a force-multiplying and/or stroke-multiplying mechanism, known in the art, may be implemented (e.g., using levers and/or pulleys) to combine the force and/or motion output of the actuating elements originated from the housing 110.

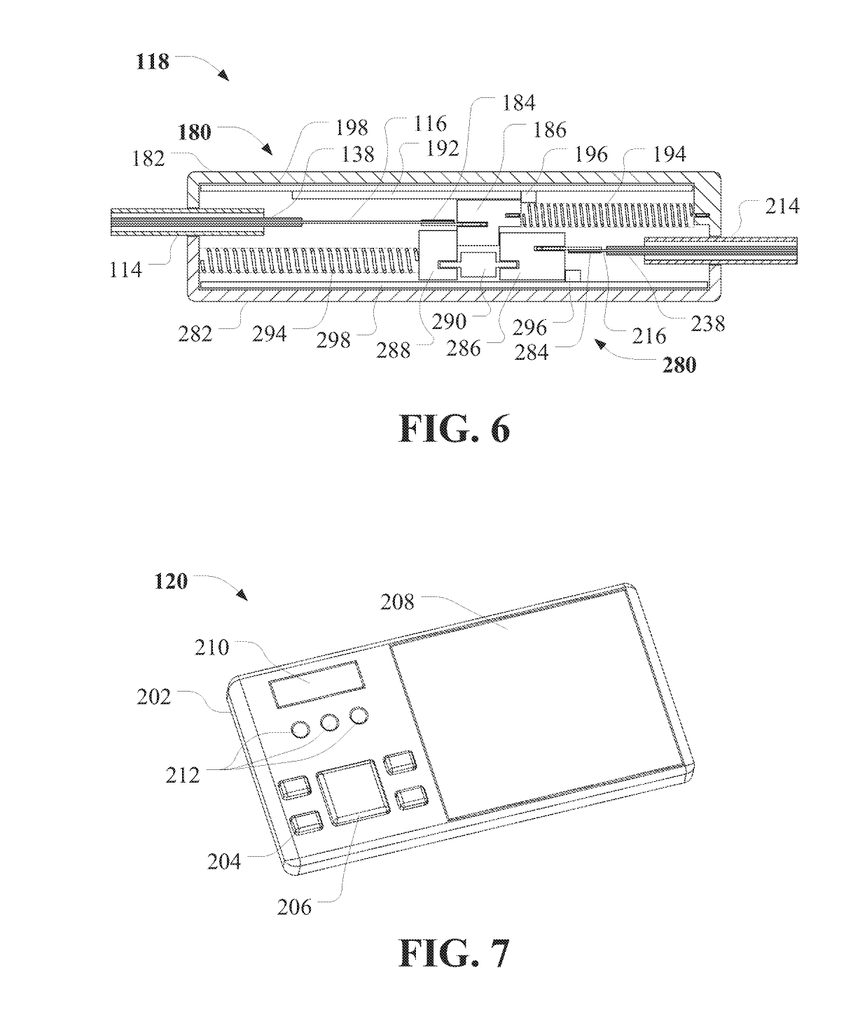

Referring to FIG. 7, an example of the user interface 120 of the actuation apparatus 100 is illustrated. The user interface 120 may include components arranged in a rigid frame 202 that is mechanically connected or integrally formed to the coupling means 118. Alternatively or additionally, the components of the user interface 120 may be arranged in different locations either worn or carried near the user.

Through this user interface 120, the user can interact with the actuation apparatus 100 and/or terminal device 102. For example, it may be used for controlling the terminal device 102, customizing settings, or conveying information to the user.

Specifically, the user interface 120 preferably comprises user input devices 156, and more specifically an array of tactile switches 204-206 (i.e., momentary push-button switches). Alternatively or additionally, the user interface 120 may comprise other types of switches, including, for example, slide, rotary, or toggle switches.

Alternatively or additionally, user input devices 156 may include one or more sensors known in the art for assessing muscle activity (e.g., EMG sensors, or pressure-sensitive pads in contact with the stump of the amputee), or brain activity of the user (e.g., EEG sensors).

Alternatively or additionally, user input devices 156 may include one or more sensors to sense user's body input. For example, motion-tracking sensors, such as accelerometers, gyroscopes, inertial measurement units (IMUs), and/or magnetometers, may be positioned in one or more locations of the actuation apparatus 100 to measure or determine the acceleration, velocity, spatial position, and/or orientation of the moving parts of the actuation apparatus 100. The outputs of these sensors may be used to enhance the overall control of the terminal device 102. For example, the user may select an operating mode of the terminal device 102 by making a predetermined motion grabbed by the motion-tracking sensors. In another example, the wrist unit 104 may be configured to automatically move to stabilize the terminal device 102 in a predetermined position and/or orientation to assist performing a task.

Alternatively or additionally, body input may be measured by other type of sensors, including but not limited to position and force sensors, well known to those skilled in the art. In further embodiments, the actuation apparatus 100 may include a global positioning system (GPS) to monitor the global position and real-time movement of the actuation apparatus 100.

Alternatively or additionally, the user interface 120 may include a speech recognition module. This module may be used for controlling the terminal device 102 using voice commands. In some preferred embodiments, a miniature microphone is installed within the rigid frame 202 while the electronic circuits for filtering and processing of the speech are installed within the housing 110.

Alternatively or additionally, the actuation apparatus 100 may be interfaced with any external input device, including, for example, a keyboard, a joystick, a 3d mouse, a touch panel, and a dataglove. For example, the amputee may use a dataglove on the sound hand for master-slave control of the terminal device 102. These external input devices may either interface directly to the actuation apparatus 100 or through a terminal device (e.g., a personal computer).

In preferred embodiments, the user interface 120 further comprises user feedback devices 158, including a display module 208, a speaker 210, and one or more status indicators 212.

The display module 208 may be of any known type (e.g., LCD, OLED, touchscreen) and can be used to display a variety of information to the user.

The speaker 210 may be of any known type and can be used to provide auditory feedback to the user. The auditory signals may include, for example, alarms, buzzers, beepers, whistles, or sirens. Although not illustrated, the actuation apparatus 100 may further comprise a connection jack to output any audio signals to an earphone or a headphone.

The status indicators 212 may be light emitting diodes (LEDs) or any other light sources, as known to those skilled in the art, for displaying information to the user. For example, they may be used to communicate to the user—through simple illumination, flashing patterns, color patterns, or other lighting effects—the operating mode of the actuation apparatus 100 and/or terminal device 102, faults, alerts, and/or the like. To this scope, the user interface 120 may include a plurality of status indicators 212 arranged at multiple locations of the actuation apparatus 100. The user interface 120 may also include a protective cover (not shown), which may preferably include one or more translucent portions in the region of status indicators 212 and display module 208.

The actuation apparatus 100 may also include suitable light sources for decorative purposes, or to increase or diminish overall visibility of the terminal device 102 or to direct light to locations in proximity to objects being grasped for increasing visual acuity and/or enhancing utility of the terminal device 102 in unfavorable lighting conditions.

In further embodiments, visual feedback may be enhanced by means of vision sensors (e.g., camera, infrared sensor, ultrasonic ranger, LIDAR) mounted on the actuation apparatus 100 and/or terminal device 102, and/or a pair of augmented reality glasses worn by the user and interfaced to the actuation apparatus 100. The augmented reality glasses may also be equipped with a stereo camera to improve the control of the terminal device 102.

Alternatively or additionally to auditory and visual feedback, the user interface 120 may include vibrotactile feedback devices, known in the art, to convey various information to the user. For example, vibration motors, known as tactors, may be placed against the skin of the user (e.g., on the arm) and vary their amplitude or frequency of vibration according to the grip force measured by tactile sensors on the terminal device 102. Other type of feedback information may also be communicated through the vibrotactile feedback system, including, for example, temperature or operating mode. Alternatively or additionally, tactile feedback may be implemented through heating devices (e.g., thermoelectric modules, also known as Peltier modules) or electrostimulating devices.

From the description above, a number of advantages of some embodiments of the present disclosure become evident. These include, without limitation, the following:
a. Owing to the extrinsically located driving devices and associated electronics, the terminal device 102 can be advantageously designed to be compact, lightweight and thus more comfortable to the user, durable, waterproof, and multifunctional having a high number of degrees of freedom.
b. The housing 110 has less strict size and weight constraints than terminal device 102, and thus it can offer the necessary space to accommodate a high number of actuators and/or more powerful actuators to drive the terminal device 102, and/or more sophisticated electronics to control the terminal device 102. Moreover, by means of the harness system 112, the user can comfortably carry the actuation apparatus 100.
c. The modularity of the design, wherein the actuation apparatus 100 can be easily separated from the terminal device 102, can be beneficial in reducing repair and upgrade costs.
d. The size can be adjusted to accommodate a wide range of users, from small children to large adults.
e. The use of actuator wires 126 can be advantageous in terms of weight, operating noise, and cost. For example, shape-memory alloy wires display an incredibly high power-to-weight ratio that can be exploited in developing lightweight yet powerful devices. Moreover, they are totally silent actuators that do not attract undue attention to the user when driving the terminal device 102. Furthermore, they are low-cost, direct-driven actuators that do not need complex, bulky, and expensive transmission systems, commonly used in conventional electric actuators.
f. The user-friendly user interface 120 can provide a plurality of input and feedback options to the user, including a plurality of sensors, which can enhance the control of the terminal device 102 and alleviate user's mental burden.
g. According to the user's needs, the actuation apparatus 100 can be body-powered, externally powered by means of actuators, or hybrid-actuated wherein body movements and actuators are both used for operating the terminal device 102. In hybrid-actuated embodiments, the user can exploit the benefits of both body-powered and externally powered technologies. For example, for grasping an object, the user can initially operate quickly and intuitively the terminal device 102 by harnessing body movements (e.g., glenohumeral flexion, scapular abduction or adduction, shoulder depression and elevation, chest expansion). The mechanical interaction between the harness system and the body-powered cable 122—whose one end is fixed in the housing 110 coupled to the harness system 112—can provide a degree of force and position feedback to the user, resulting in better control of the terminal device 102. After the initial body-powered operation of the terminal device 102, actuators can be used to provide the necessary force to perform or maintain a grip, and thus mechanically assist the user without requiring any manual intervention. The level of force-assistance to the user can be easily adjusted through the user-interface 120 and implemented by the control unit 150. For example, in a maximum level of force-assistance, the body-powered cable 122 may essentially be used for capturing body motion and controlling the operation of the actuators and/or selecting operating modes of the terminal device 102. Furthermore, if needed (e.g., due to battery discharge of the actuation apparatus 100), the terminal device 102 can still be reliably operated as a standard body-powered device.

Thus the reader will see that at least one embodiment of the present disclosure provides a more functional, comfortable, lightweight, silent, durable, reliable, user-friendly, yet affordable wearable device that can be used by persons of almost any age.

The foregoing description of preferred embodiments is merely illustrative in nature and in no way intended to be construed to limit the substance or scope of the present invention. Many more embodiments and adaptations of this invention other than those specifically described herein, as well as many variations, modifications, and equivalent arrangements, will be apparent to those skilled in the art, without departing from the inventive concepts as set forth in the appended claims and their legal equivalents.

Additionally, although the preferred embodiments have been described in the context of a prosthetic hand for a unilateral transradial amputee, the actuation apparatus described herein may be easily adapted to be used by upper-limb amputees with any level of unilateral or bilateral amputation.

Furthermore, the concepts, spirit, and the scope of the present invention, illustrated in the preferred embodiments, apply equally well to other assistive terminal devices, including prosthetic, orthotic, empowering, and other assistive upper-limb devices. For example, an actuation apparatus according to the elements of this invention can be used for actuating a prosthetic hook or gripper, an exoskeleton hand, an empowering glove, or a robotic hand acting as user's third hand.

Additionally, although this disclosure has been described with regard to wearable upper-limb devices, those of ordinary skill in the art will understand and appreciate that trivial changes may readily transfer the use to other assistive, adaptive, rehabilitative, or generally robotic devices.

The inventive subject matter, therefore, is not to be restricted except in the spirit of the disclosure. Moreover, in interpreting the disclosure, all terms should be interpreted in the broadest possible manner consistent with the context. Furthermore, as used in the specification and the appended claims, the singular forms “a”, “an”, and “the” include plural referents unless the context clearly dictates otherwise.

What is claimed is:

1. An actuation apparatus for a wearable terminal device, comprising:
a housing;
a harness system coupled to the housing;
a control unit;
a movable actuating element selected from the group consisting of:
a body-powered cable,
an artificial tendon coupled to an actuator device installed in the housing, and
an actuator wire made of a material selected from the group of smart materials,
wherein one end of the actuating element is disposed within the housing;
a conduit containing the actuating element; and
coupling means for connecting the other end of the actuating element to the terminal device,
whereby the actuating element drives the terminal device.

2. The actuation apparatus of claim 1, wherein the coupling means comprise a first connector assembly and a second connector assembly interlocked to the first connector assembly.

3. The actuation apparatus of claim 1, wherein the housing comprises a length-adjusting mechanism for altering the setup position of the actuating element and the conduit.

4. The actuation apparatus of claim 1, further comprising at least one feedback sensor for the actuating element, selected from the group consisting of position and tension sensors.

5. The actuation apparatus of claim 1, further comprising a user interface, including at least one item selected from the group consisting of user input and feedback devices.

6. The actuation apparatus of claim 1, wherein the movable actuating element is enclosed by a low-friction flexible tube, and wherein the tube is enclosed by the conduit.

7. The actuation apparatus of claim 1, wherein the actuator wire is made of a shape-memory material.
